(19) United States
(12) Patent Application Publication
(10) Pub. No.: US 2011/0301559 A1
(43) Pub. Date: Dec. 8, 2011

(54) DISPOSABLE BODILY FLUID ABSORBENT WEARING ARTICLE

(75) Inventors: Kenichi Sasayama, Kagawa (JP); Takayoshi Konishi, Kagawa (JP)

(73) Assignee: UNI-CHARM CORPORATION, Ehime (JP)

(21) Appl. No.: 13/147,462

(22) PCT Filed: Feb. 3, 2010
(86) PCT No.: PCT/JP2010/051513

§ 371 Date: Aug. 2, 2011

(30) Foreign Application Priority Data
Feb. 6, 2009 (JP) 2009-026719

(51) Int. Cl. A61F 13/58; A61L 15/62

(52) U.S. Cl. 604/364; 604/385.03

(57) ABSTRACT

The present invention provides a disposable bodily fluid absorbent wearing article so that the article can be easily fitted to a desired region of a wearer's body. A disposable bodily fluid absorbent wearing article 1 is formed by bending a flat pad along the center line L thereof with a liquid-pervious skin-contactable sheet 2 facing outward so that a cross section taken in a transverse direction B may be invert V-shaped. In the bodily fluid-absorbent wearing article 1, respective halves of a garment-contactable sheet 3 are partially bonded to each other by means of bonding means 11 so that the posture of the pad bent in the inverted V-shape may be maintained. The garment-contactable sheet 3 is provided with fastening means 12 by which the bodily fluid absorbent wearing article 1 can be detachably fastened to the wearer's garment.

### TECHNICAL FIELD

The present invention relates to disposable bodily fluid absorbent wearing articles suitable to be put on a wearer's crotch skin and, more particularly, to disposable bodily fluid absorbent wearing articles suitable to be used as hemorrhoid pad, labial pads or the like.

### BACKGROUND

Conventionally, disposable wearing articles adapted to be aligned with and put against a target portion in the crotch region of a wearer, such as the female genital organs, to absorb bodily fluids are known. Articles of such kind particularly being formed so that the midsection in the width direction become higher than the peripheral region are also known.

For example, JP 1999-313851 A1 (PTL 1) discloses a sanitary napkin having an inverted V-shaped cross section taken in a width direction. This sanitary napkin has a lower surface of a flat backsheet member being coated with pressure-sensitive adhesives and extending outward in the width direction beyond opposite side edges of an inverted V-shaped core. In use, this sanitary napkin is fastened to the wearer's undergarment with the pressure-sensitive adhesives.

JP 2007-111191 A1 (PTL 2) discloses a labial pad having an inverted V-shaped cross section taken in a width direction. In this pad, a connector member made of elastic material extends in the width direction and is bonded to flaps defined on the outside of transversely opposite side edges of an inverted V-shaped absorbent core.

### Citation List

### {Patent Literature}

{PTL 1} JP 1999-313851 A1

{PTL 1} JP 2007-111191 A1

### SUMMARY

### Technical Problem

For these wearing articles of known art having the inverted V-shaped cross section taken in the width direction, it is preferable, in actual use, to fit the apex of the inverted V-shaped cross section accurately to the target region of the wearer's crotch. However, in the sanitary napkin disclosed by PTL 1, once the release paper covering the pressure-sensitive adhesives has been peeled off, the pressure-sensitive adhesives comes closest to the inner surface of the wearer's undergarment and readily comes in contact with the undergarment. After the pressure-sensitive adhesives have been put in contact with the inner surface of the undergarment, it is difficult to adjust a position of the sanitary napkin relative to the inner surface of the undergarment.

The labial pad disclosed by PTL 2 claims that, once the pad has been put between vaginal lips, the connector member made of an elastic material reliably prevents the pad from being displaced out of alignment with the vaginal lips under the repelling force of the connector means. However, the stability of the pad between the vaginal lips largely depends on initial positioning of the pad and that the pad is likely to be readily displaced from the vaginal lips.

An object of the present invention is to improve a disposable bodily fluid absorbent wearing article so that the article can be easily fitted to a desired region of the wearer's body.

### Solution to Problem

According to the present invention, there is provided a disposable bodily fluid absorbent wearing article having a longitudinal direction, a transverse direction and a thickness direction being orthogonal one to another, and comprising a liquid pervious skin-contactable sheet, a garment-contactable sheet and a bodily fluid absorbent core sandwiched between the skin-contactable sheet and the garment-contactable sheet in the thickness direction so as to form a flat pad being symmetric about a centerline extending in the longitudinal direction and bisecting a dimension of the pad in the transverse direction wherein the flat pad is bent in two halves along the centerline with the skin-contactable sheet facing outward so as to define an inverted V-shaped cross section.

The improvement according to the present invention is characterized as follows: the respective halves of the garment-contactable sheet facing each other in the transverse direction about the centerline are partially connected to each other by connector means so that the pad may be maintained in the state of being bent in the inverted V-shape, and the bodily fluid absorbent wearing article is provided with fastening means adapted for detachably fastening of the garment-contactable sheet to the wearer's garment.

The invention includes an embodiment wherein the fastening means comprise adhesives applied to portions of the respective halves of the garment-contactable sheet facing each other or a connector sheet having both laterals in the transverse direction attached to portions of the respective halves of the garment-contactable sheet.

The invention includes an embodiment wherein the skin-contactable sheet and the garment-contactable sheet extend outward beyond a peripheral edge of the core and are layered and bonded together along respective extending portions to define a peripheral region of the bodily fluid absorbent wearing article, and the fastening means are formed inside the peripheral region of the bodily fluid absorbent wearing article.

The invention includes an embodiment wherein the flat pad has a contour line defining a planar shape thereof and the contour line includes convexly curved segments so as to be furthest from the center line and the flat pad is formed inside the convexly curved segments with the fastening means.

The invention includes an embodiment wherein the fastening means are formed of one of a pressure-sensitive adhesive and a hook member of a mechanical fastener.

The invention includes an embodiment wherein the skin-contactable sheet, the garment-contactable sheet and the connector sheet are respectively formed of water-disintegrable sheets and the core is a mass of liquid-absorbent fibers each having a fiber length of 20 mm or less.

The invention includes an embodiment wherein any one of the skin-contactable sheet, the garment-contactable sheet and the connector sheet is formed of a water-disintegrable nonwoven fabric containing ultrafine thermoplastic fibers each having a fineness in a range of 0.01 to 0.5 dtex and a fiber length in a range of 3 to 10 mm in a range of 10 to 50% by mass.

The invention includes an embodiment wherein the connector means and the fastening means are spaced from each other in the longitudinal direction.

The invention includes an embodiment wherein the connector means is formed on only one end portion of two end portions of the bodily fluid absorbent wearing article opposed to each other in the longitudinal direction.

### Advantageous Effects of Invention

In the disposable bodily fluid absorbent wearing article according to the present invention, any one of the garment-contactable sheet and the connector sheet attached thereto is provided with the fastening means adapted for detachably fastening of the bodily fluid absorbent wearing article to the wearer's garment. With such arrangement, when this article is used for a hemorrhoidal patient in close alignment with anus, for example, the article can be fastened to the patient's garment such as pants with this fastening means. In this way, the article should not be displaced out of alignment with the anus even when the patient moves his or her body.

### BRIEF DESCRIPTION OF DRAWINGS

FIG. 1 Partially cutaway perspective view of a disposable bodily fluid absorbent wearing article (hemorrhoid pad).

FIG. 2 Sectional view taken along line II-II in FIG. 1.

FIG. 3 Sectional view taken along line III-III in FIG. 1.

FIG. 4 Partially cutaway plan view showing the bodily fluid absorbent wearing article in FIG. 1 as has been flatly developed.

FIG. 5 View similar to FIG. 4, showing one embodiment of the invention.

FIG. 6 View similar to FIG. 1, showing another embodiment of the invention.

FIG. 7 Sectional view taken along line VII-VII in FIG. 6.

FIG. 8 Sectional view taken along line VIII-VIII in FIG. 6.

FIG. 9 Partially cutaway plan view showing the bodily fluid absorbent wearing article in FIG. 6 as has been flatly developed.

### DESCRIPTION OF EMBODIMENTS

Details of a disposable bodily fluid absorbent wearing article according to the invention will be more fully understood from the description given hereunder with reference to the accompanying drawings.

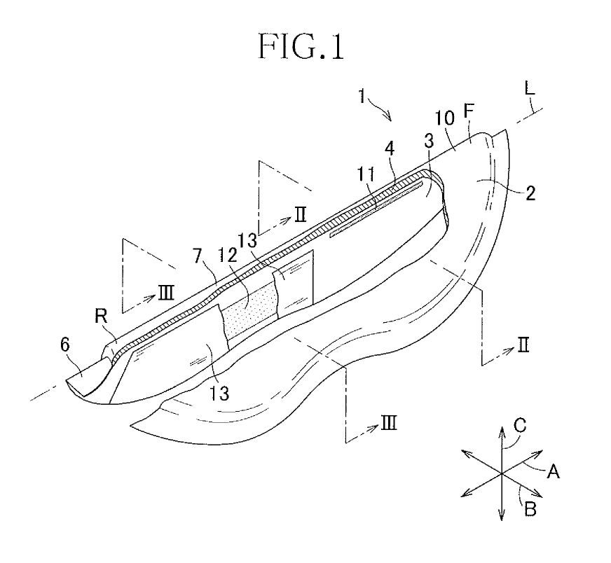

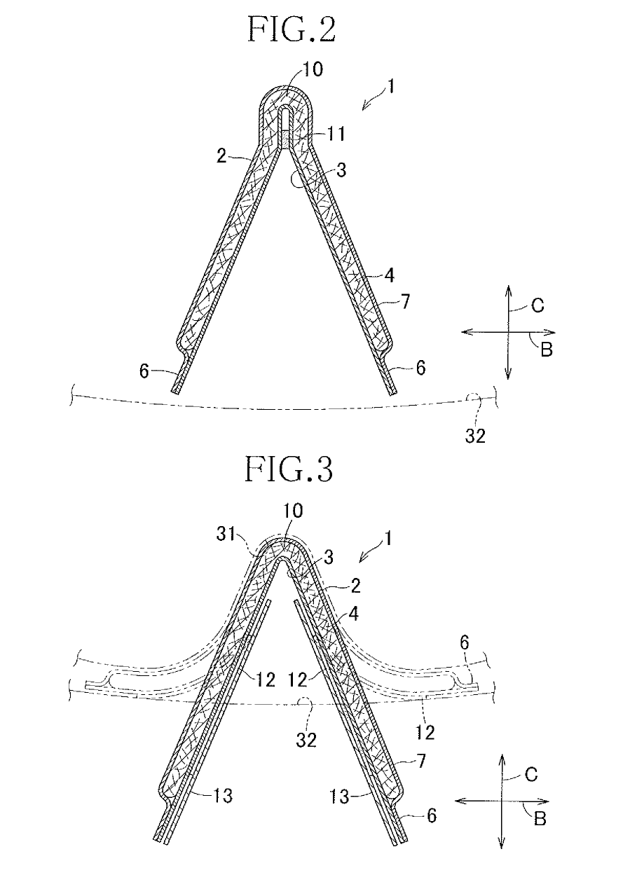

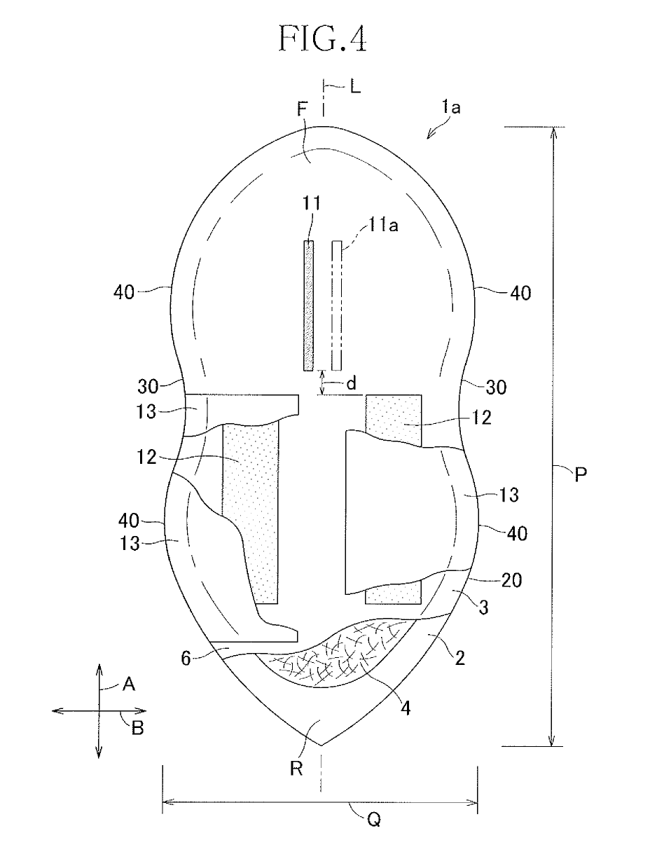

FIGS. 1, 2 and 3 are the partially cutaway perspective view of a hemorrhoid pad 1 as an example of the disposable bodily fluid absorbent wearing article, the sectional view taken along line II-II in FIG. 1 and the sectional view taken along line III-III in FIG. 1, respectively. The pad 1 has a longitudinal direction A, a transverse direction B and a height direction C being orthogonal one to another and includes a front end portion F and a rear end portion R opposed to each other in the longitudinal direction A. The pad 1 is shaped symmetrically about a center line L bisecting a dimension in the transverse direction B. Referring to the sectional views of FIGS. 2 and 3, the pad 1 is bent in two along the center line L so as to define an inverted V-shape having an apex at the highest level in the height direction C. Referring to FIG. 1, the apex 10 comes into line with the center line L and extends in the longitudinal direction A.

The pad 1 includes a skin-contactable sheet 2, a garment-contactable sheet 3 and a bodily fluid absorbent core 4 sandwiched between these two sheets 2, 3. The skin-contactable sheet 2 is liquid-pervious and the garment-contactable sheet 3 may be liquid-impervious or liquid-pervious, preferably, liquid-impervious. Both the skin-contactable sheet 2 and the garment-contactable sheet 3 extend outward beyond a peripheral edge of the core 4 and are layered and bonded together along respective extending portions with adhesives or by fusing thermoplastic synthetic resins contained in these sheets 2, 3 to define a peripheral region 6 of the pad 1. In the preferred peripheral region 6, the skin-contactable sheet 2 and the garment-contactable sheet 3 are bonded to each other particularly with water-soluble adhesives such as starch pastes. The inner side area surrounded by the peripheral region 6 defines a bodily fluid absorbent region 7.

In the inverted V-shaped section shown in FIGS. 2 and 3 to be taken in the transverse direction B, the skin-contactable sheet 3 defines an outer surface of the pad 1 and the garment-contactable surface 4 defines an inner surface of the pad 1. In FIG. 2 showing the section of the pad 1 taken in the front end portion F of the pad 1, respective halves of the garment-contactable sheet 3 facing each other about the center line L (See FIG. 1) are bonded to each other in the vicinity of the apex 10 with adhesives 11. In FIG. 3 showing the section of the pad 1 taken in the rear end portion R of the pad 1, the respective halves of the garment-contactable sheet 3 facing each other about the center line L are coated with pressure-sensitive adhesives 12 which are protectively covered with release sheets but not bonded to each other with the adhesives 11 as in the case of FIG. 2.

FIG. 4 is a partially cutaway plan view showing the pad 1 flatly developed after the respective halves of the garment-contactable sheet 3 bonded together with the adhesives 11 as seen in FIG. 2 have been peeled off from each other. While such flatly developed pad 1a is symmetric about the center line L, such symmetric relationship is not applicable to the adhesives 11. Specifically, the adhesives 11 are shown on one side of the center line L and the region to be bonded to the counterpart with the adhesives 11 are indicated on the other side of the center line L by an imaginary line 11a. The adhesives 11 are applied to the garment-contactable sheet 3 only on the side of the front end portion F and most part of the pressure-sensitive adhesives 12 are applied to the garment-contactable sheet 3 on the side of the rear end portion R opposite to the front end portion F. The adhesives 11 and the pressure-sensitive adhesives 12 are spaced at a distance d from each other in the longitudinal direction A. While a planar shape of the flat pad 1a is not specified according to the present invention, a contour line 20 defining the planar shape of the flat pad 1a alternately curve convexly and concavely on each side of the center line L according to the illustrated embodiment so that convex curve lines are formed on a front end 21 and a rear end 22 of the flat pad 1a. Respective segments of the contour line 20 defined on both sides of the center line L include concavely curved segments 30 and convexly curved segments 40 at the furthest distance from the center line L. In the flat pad 1a exemplarily illustrated, the pressure-sensitive adhesives 12 are applied in zones defined inside the respective convexly curved segments 40 inboard of the peripheral region 6. The longitudinal direction A, the transverse direction B and the center line L of the flat pad 1a formed in this manner correspond to the longitudinal direction A, the transverse direction B and the center line L in FIG. 1, respectively. The height direction C in FIG. 1 corresponds to the thickness direction (not shown) of the flat pad 1a in FIG. 4. The thickness direction of this flat pad 1a is orthogonal to the longitudinal direction A and the transverse direction B in FIG. 4. While a dimension P in the longitudinal direction A and a dimension Q in the transverse direction B of the flat pad 1a may be selected depending on a physical size of the wearer, a commonly used hemorrhoid pad 1 has the dimension P in a range of 8 to 15 cm and the dimension Q in a range of 4 to 9 cm.

The pad 1 may be put on the wearer's body, for example, in a sequence as follows: the pad 1 as a whole is inserted into bottom cleavage 31 with the apex 10 in the front end portion F aligned with and put against anus of the hemorrhoidal patient as indicated by imaginary lines in FIGS. 2 and 3. In the course of insertion of the pad 1 in this manner, a finger may be inserted into a space defined between the respective halves of the garment-contactable sheet 3, 3 facing each other so as to define the inverted V-shape in the rear end portion R of the pad 1 to facilitate the operation of insertion. The release sheet 13 may be peeled off before the pad 1 is inserted into the bottom cleavage 31 or after the pad 1 has been aligned with and put against the anus of the wearer. With the pad 1 put against anus, pants 32 into which the wearer has already stepped may be fully pulled up to put the inner surface of the pants 32 in close contact with the pressure-sensitive adhesives 12 and thereby to fasten the pad 1 to the inner surface. Thereupon, a region of the pad 1 including the pressure-sensitive adhesives 12 is deformed along the inner surface of the pants 32. While the rear end portion R of the pad 1 is deformed in this manner, the front end portion F not fastened to the pants 32 is kept in its inverted V-shape, i.e., its angular shape and apt to be biased in the opposite direction of the rear end portion R. Specifically, the front end portion F is apt to be raised from the inner surface of the pants 32 toward the anus, restricting a possibility that the pad 1 might come off from the bottom cleavage. Such behavior of the front end portion F advantageously functions to accelerate absorption of bodily fluids. In such situation of the front end portion F, the pants 32 and the peripheral region 6 are spaced from each other in the height direction C as indicated by imaginary lines in FIG. 2.

Such manner of wearing the pad 1 assures that the pressure-sensitive adhesives 12 applied to the pad 1 inside the peripheral region 6 is kept out of contact with the inner surface of the pants 32 regardless of the time point at which the release sheet 13 is peeled off and unless the inner surface of the pants 32 is pressed against the pressure-sensitive adhesives 12. More specifically, the pressure-sensitive adhesives 12 should not come in contact with the inner surface of the pants 32 in the course of pulling up of the pants 32. The pad 1 can be fastened to the inner surface of the pants 32 at the appropriate position thereof without any difficulty. After the pad 1 has been fastened to the inner surface of the pants 32 with the pressure-sensitive adhesives 12, the pad 1 should not be readily displaced out of alignment with the wearer's anus (not shown) even when he or she moves. Tack strength of the pressure-sensitive adhesives 12 is adjusted so that the pad 1 may be easily peeled off from the inner surface of the pants 32 and, in view of this, the pressure-sensitive adhesives 12 may be preferably selected from a group of water-soluble products such as a pressure-sensitive adhesives made of polyvinyl alcohol.

The pad 1 may be put on the wearer's body in an alternative sequence as follows: The pad 1 is fastened to the inner surface of the pants 32 at the appropriate position before the pad 1 is aligned with and put against the wearer's anus. A posture of the pad 1 in this step is indicated by imaginary lines in FIG. 3. The front end portion F of the pad 1 has its apex 10 maintained convex under the effect of the adhesives 11 and therefore the front end portion F is smoothly moved into the wearer's bottom cleavage 31 as the pants worn in a halfway manner are pulled up. For a patient using regularly the pad 1, the target region in which the pad 1 should be fastened to the pants 32 becomes constant and it is easy for such wearer to know where such target region is.

In the pad 1, the skin-contactable sheet 2 may be formed, for example, by a liquid-pervious nonwoven fabrics or woven fabrics, or a perforated plastic films. The garment-contactable sheet 3 may be formed, for example, of liquid-pervious or liquid-impervious nonwoven fabrics or woven fabrics, or plastic films. However, liquid-impervious materials are preferably used in order to prevent any quantity of bodily fluids once having been absorbed by the pad 1 from leaching out from the pad 1. The core 4 may be formed of fluff pulp or a mixture of the fluff pulp and super-absorbent polymer particles.

The skin-contactable sheet 2 and the garment-contactable sheet 3 may be formed of a water-disintegrable nonwoven fabric of which tissue in the form of a sheet is disintegrated into a plurality of fractions when this nonwoven fabric is stirred in a large amount of water. As an example of such nonwoven fabric, there are spun laced nonwoven fabrics including ultrafine thermoplastic synthetic fibers each having a fineness in a range of 0.01 to 0.5 dtex and a fiber length in a range of 3 to 10 mm in 10 to 50% by mass, chemical fibers each having a fineness in a range of 1 to 2 dtex and a fiber length in a range of 5 to 20 mm and/or pulp fibers having a freeness in a range of 600 to 770 cc in 90 to 50% by mass. More specifically, there is a spun laced nonwoven fabric having a basis mass of 35 g/m² and a thickness of 0.24 mm including 0.3 dtex ultrafine thermoplastic synthetic fibers in 20 % by mass which are obtained by splitting a polyethylene terephthalate/nylon conjugate fibers each having a fineness of 3.3 dtex and a fiber length of 5 mm and being formed to be splittable into 11 fibers, and rayon fibers having a fineness of 1.1 dtex and a fiber length of 7 mm in 80% by mass. The spun laced nonwoven fabric is water-disintegrable and has a sufficient tensile strength, flexibility and liquid-perviousness to be used as the skin-contactable sheet 2. This nonwoven fabric may be coated with a water-soluble binder such as AQ55S produced by Eastman Chemical Company in 8% by mass of a total mass of the nonwoven fabric to obtain the nonwoven fabric which is water-disintegrable but sufficiently low in liquid-perviousness to be used as the garment-contactable sheet 3. In the pad 1 using the water-disintegrable nonwoven fabric, the core 4 is preferably formed of liquid-absorbent fibers such as pulp fibers having a fiber length of 20 mm or less including none of super-absorbent polymer particles so that the core 4 also may be disintegrated into fractions when stirred in a large amount of water. This is because the super-absorbent polymer particles contained in the core 4 may form a water-insoluble block as the core 4 absorbs liquid.

While either water-soluble adhesives or hot melt adhesives may be used as the adhesives 11, it is preferred to use the water-soluble adhesives from the viewpoint of improvement in the water-disintegration of the pad 1. If the garment-contactable sheet 3 of the pad 1 contains thermoplastic resin, the respective halves of the garment-contactable sheet 3 bent in the inverted V-shape may be locally fusion-bonded instead of bonding with the adhesives 11. In either case, it is preferable to constrict the area in which the respective halves of the garment-contactable sheet 3 are locally bonded together in order to prevent the water-disintegrable property of this sheet 3 from being deteriorated.

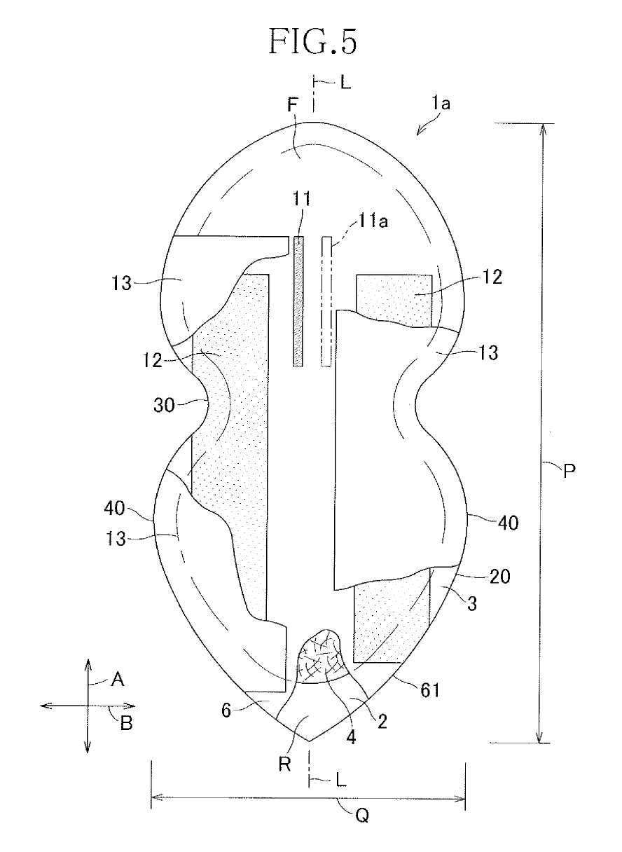

FIG. 5 is a view similar to FIG. 4, showing one embodiment. The flatly developed pad 1a according to this embodiment is distinguished from the flatly developed pad 1a in FIG. 4 not only in its planar shape but also in the location of the pressure-sensitive adhesives 12. Specifically, the pressure-sensitive adhesives 12 extends into the front end portion F so as to be partially lateral to the adhesives 11. It is possible to extend a range of coating with the adhesives 12 in such a manner. In this embodiment, the pressure-sensitive adhesives 12 are applied to the inner surface of the garment-contactable sheet 3 inside the convexly curved segments 40 of the contour line 20 but the outermost edge of the pressure-sensitive adhesives 12 come into line with the contour line 20 along the concavely curved segments and in regions 61 of the rear end portion R defined in the vicinity of the center line L. In principle, the pressure-sensitive adhesives 12 should be applied to the garment-contactable sheet 3 inside the convexly curved segments 40 of the contour line 20. However, the pressure-sensitive adhesives 12 may be applied to the segments of the contour line 20 which will not come in contact with the inner surface of the pants 32 when the pad 1 is erected on the inner surface of the pants 32 in the inverted V-shape as viewed in FIGS. 2 and 3. When the pad 1 corresponding to the flatly developed pad 1a of FIG. 5 is fastened to the inner surface of the pants 32, not only the rear end portion R but also the front end portion F can be fastened to the inner surface of the pants 32 and thereby it is assured to fasten the pad 1 to the pants 32 tightly. With regard to the pad 1 according to this embodiment, also, the pressure-sensitive adhesives 12 should not come in contact with the inner surface of the pants 32 even if the release sheet 13 has been peeled off.

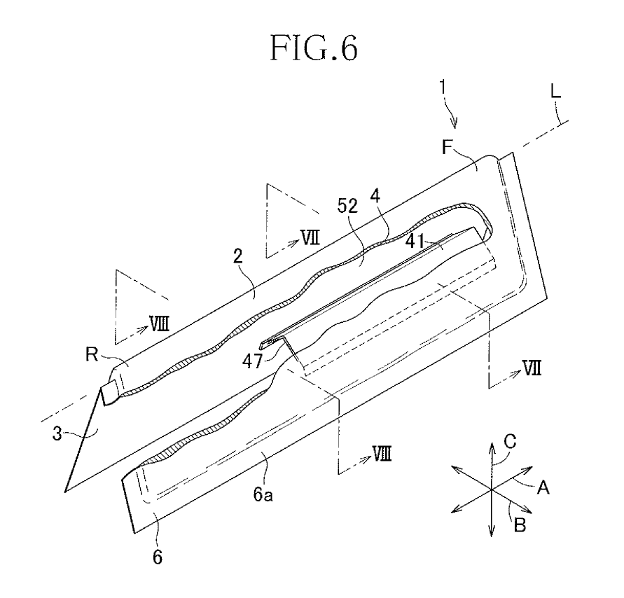

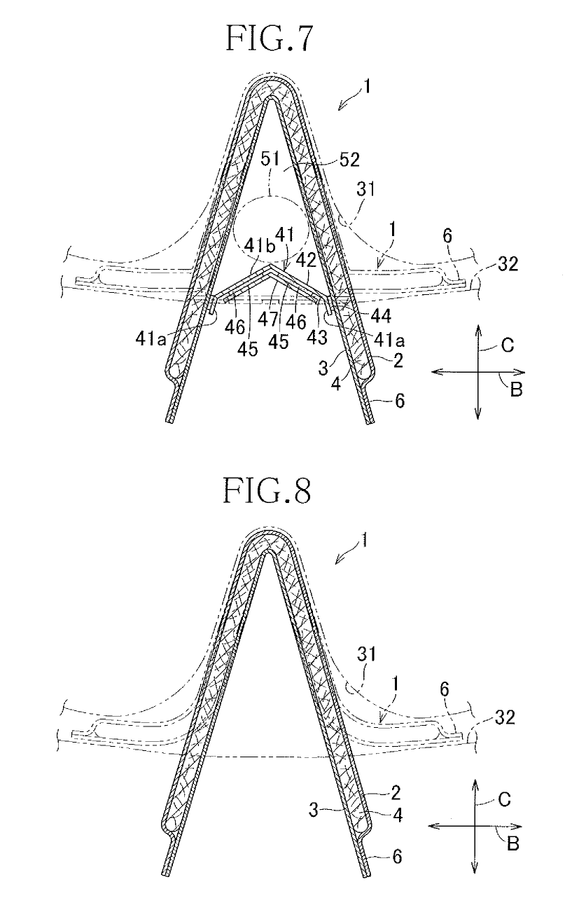

FIGS. 6, 7 and 8 are views similar to FIGS. 1, 2 and 3, respectively, showing another embodiment wherein FIG. 6 is a partially cutaway perspective view showing the hemorrhoidal pad 1, FIG. 7 is a sectional view taken along line VII-VII in FIG. 6 and FIG. 8 is a sectional view taken along line VIII-VIII in FIG. 6. The pad 1 shown in FIGS. 6 and 7 is distinguished from the pad 1 shown in FIG. 1 in that the respective halves of the garment-contactable sheet 3 in the front end portion F of the pad 1 facing each other in the transverse direction B about the center line L are connected with each other by means of a bridge-like connector sheet 41. The connector sheet 41 is symmetric about the center line L and has laterals 41a opposite to each other in the transverse direction B have respective surfaces 42 facing the garment-contactable sheet 3 permanently bonded to the garment-contactable sheet 3 with adhesives 44. The connector sheet 41 has an intermediate section 41b extending between the laterals 41a and free from the garment-contactable sheet 3 and a lower surface 43 of the intermediate section 41b opposite to its upper surface 42 is formed symmetrically about the center line L with a pair of pressure-sensitive adhesive regions 45 spaced from each other in the transverse direction B by means of which the lower surface 43 is detachably fastened to the pants as a garment 32 (See FIG. 7). These pressure-sensitive adhesive regions 45 respectively contain pressure-sensitive adhesives 46 serving as fastening means by means of which the pad 1 is temporarily fastened to the garment 32, and the pressure-sensitive adhesives 46 are protectively covered with a release sheet 47 extending in the transverse direction B in FIG. 7. The positions at which the laterals 41a of the connector sheet 41 are bonded to the garment-contactable sheet 3 are previously selected so that the intermediate section 41b of the connector sheet 41 may be positioned above the peripheral region 6 of the pad 1 in the height direction C when the pad 1 is erected in the inverted V-shape as shown in FIG. 7. The preferred laterals 41a of the connector sheet 41 are bonded to the garment-contactable sheet 3 above the peripheral region 6 as exemplarily shown in FIG. 7. The pressure-sensitive adhesive regions 45 in the connector sheet 41 lie inside the peripheral region 6 of the pad 1 in the transverse direction B and above the peripheral region 6 in the height direction C. As will be apparent from FIG. 8, the rear end portion R of the pad 1 has the same configuration as the rear end portion R of the pad 1 shown in FIG. 2.

In the pad 1 constructed in the manner as has been described above, the connector sheet 41 functions in the same fashion as that of the adhesives 11 shown in FIG. 2 to prevent the inverted V-shaped pad 1 from excessively expanding in the transverse direction B and thereby to maintain the pad 1 in the properly bent posture. To put the pad 1 on the wearer's body, the finger 51 indicated by an imaginary line in FIG. 7 may be inserted from the end of the rear end portion R of the pad 1 into a space defined between the respective halves of the garment-contactable sheet 3 facing to each other so that the finger 51 may be inserted into a space 52 defined between the garment-contactable sheet 3 and the connector sheet 41. In this way, the pad 1 is captured by the finger 51 and easily brought in close alignment with the wearer's anus. The release sheet 97 may be peeled off before the finger 51 is inserted into the space 52 of after the pad 1 has been brought in close alignment with the anus. In any case, the pad 1 should not be unintentionally bonded to the inner surface of the pants 32 even when the peripheral region 6 of the pad 1 may come in contact with the pants 32 in the course of pulling the pants up. This is because the pressure-sensitive adhesives 46 lies inside and above the peripheral region 6 of the pad 1. The posture of the pad 1 fastened to the pants 32 after the pants 32 have been fully pulled up is indicated by imaginary lines in FIGS. 7 and 8. As shown, the portion of the pad 1 in the vicinity of the peripheral region 6 is deformed along the inner surface of the pants 32.

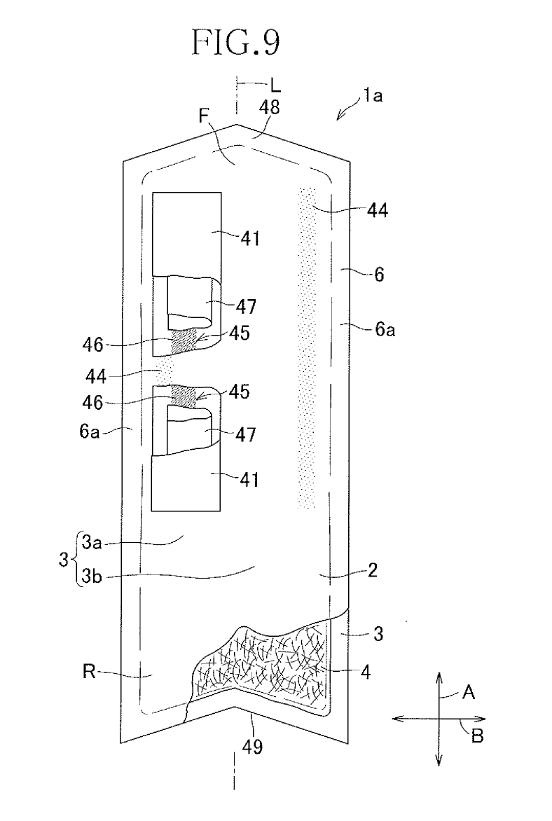

FIG. 9 is a partially cutaway plan view of a flat pad 1a according to another preferred embodiment obtained, in the same manner as in the case of FIG. 4, by flatly developing the skin-contactable sheet 2, the garment-contactable sheet 3 and the core 4 collectively bent along the center line L. The flat pad 1a according to this embodiment has a shape different from the shape exemplarily shown in FIG. 4. The peripheral region 6 includes segments 6a extending in parallel to the center line L, the end of the front end portion F extends outwardly of the flat pad 1a at a sharp angle so as to define a convex end 48 and the end of the rear end portion R extends inwardly of the flat pad 1a at the same angle as the sharp angle of the convex end 48 so as to define a concave end 49. The flat pad 1a shaped in this manner is advantageous from the viewpoint that a quantity of scraps left behind in the course of making the respective members including the garment-contactable sheet 2 can be reduced. It should be noticed that the connector sheet 41 is folded back on itself together with the release sheet 47 and the connector sheet 41 is bonded with adhesives 44 to a section 3a of the garment-contactable sheet 3 lying on one side of the center line L. The garment-contactable sheet 3 is folded back along the center line L and its section 3b lying on the other side of the center line L is lapped over the connector sheet 41 and bonded thereto with the adhesives 44. The pad 1 is obtained by folding such flat pad 1a in two.

While the connector sheet 41 in the pad 1 shown in FIG. 6 may be formed of sheet materials such as nonwoven fabrics, woven fabrics, paper or plastic films, the preferred connector sheet 41 is formed of water-disintegrable sheet materials. With regard to the connector sheet 41, the pressure-sensitive adhesives 46 pair of pressure-sensitive adhesive regions 45 arranged to be spaced from each other in FIG. 7 may be replaced by the pressure-sensitive adhesives 46 coated so that a pair of pressure-sensitive adhesive regions 45 may become contiguous to each other in the transverse direction B.

The pressure-sensitive adhesives 12 in FIG. 2 and the pressure-sensitive adhesives 46 in FIG. 7 both serving as means to fasten the pad 1 to the pants 32 may be replaced by hook member of a fastener comprised of the hook member and the loop member adapted to be detachably engaged with each other and widely known in the name of Magic Tape (trademark) wherein the inner surface of the pants 32 is used as the loop member which is a counterpart of the hook member.

The present invention having been exemplarily described above with respect to the hemorrhoid pad 1 is applicable also to the other types of the bodily fluid absorbent wearing article such as a disposable labial pad.

### REFERENCE SIGNS LIST

1 bodily fluid absorbent wearing article (hemorrhoid pad)
1a flatly developed pad
2 skin-contactable sheet
3 garment-contactable sheet
4 core
6 peripheral region
11 bonding means (adhesives)
12 fastening means (pressure-sensitive adhesives)
20 contour line
40 convexly curved segments
41 bonding means (connector sheet)
46 fastening means (pressure-sensitive adhesives)
A longitudinal direction
B transverse direction
L center line

1. A disposable bodily fluid absorbent wearing article having a longitudinal direction, a transverse direction and a thickness direction being orthogonal one to another, and comprising a liquid pervious skin-contactable sheet, a garment-contactable sheet and a bodily fluid absorbent core sandwiched between the skin-contactable sheet and the garment-contactable sheet layered in the thickness direction so as to form a flat pad being symmetric about a center line extending in the longitudinal direction and bisecting a dimension of the pad in the transverse direction wherein the flat pad is bent in two halves along the center line with the skin-contactable sheet facing outward so as to define an inverted V-shaped cross section, characterized in that:
the respective halves of the garment-contactable sheet facing each other in the transverse direction about the center line are partially connected to each other by connector means so that the pad may be maintained in a state of being bent in the inverted V-shape and the bodily fluid absorbent wearing article is provided with fastening means adapted for detachably fastening of the garment-contactable sheet to the wearer's garment.

2. The bodily fluid absorbent wearing article defined by claim 1, wherein the fastening means comprise adhesives applied to portions of the respective halves of the garment-contactable sheet facing each other or a connector sheet having both laterals in the transverse direction attached to portions of the respective halves of the garment-contactable sheet.

3. The bodily fluid absorbent wearing article defined by claim 1, wherein the skin-contactable sheet and the garment-contactable sheet extend outward beyond a peripheral edge of the core and put flat and bonded together along respective extending portions to define a peripheral region of the bodily fluid absorbent wearing article and the fastening means are formed inside the peripheral region.

4. The bodily fluid absorbent wearing article defined by claim 1, wherein the flat pad has a contour line defining a planar shape thereof and the contour line includes convexly curved segments so as to be furthest from the center line and the flat pad is formed inside the convexly curved segments with the fastening means.

5. The bodily fluid absorbent wearing article defined by claim 1, wherein the fastening means are formed of one of a pressure-sensitive adhesive and a hook member of a mechanical fastener.

6. The bodily fluid absorbent wearing article defined by claim 1, wherein the skin-contactable sheet, the garment-contactable sheet and the connector sheet are respectively formed of water-disintegrable sheets and the core is an assembly of liquid-absorbent fibers each having a fiber length of 20 mm or less.

7. The bodily fluid absorbent wearing article defined by claim 1, wherein any one of the skin-contactable sheet, the garment-contactable sheet and the connector sheet is formed of water-disintegrable nonwoven fabric containing ultrafine thermoplastic fibers each having a fineness in a range of 0.01 to 0.5 dtex and a fiber length in a range of 3 to 10 mm in a range of 10 to 50% by mass.

8. The bodily fluid absorbent wearing article defined by claim 1, wherein the connector means and the fastening means are spaced from each other in the longitudinal direction.

9. The bodily fluid absorbent wearing article defined by claim 1, wherein the connector means is formed on only one end portion of two end portions of the bodily fluid absorbent wearing article opposed to each other in the longitudinal direction.
